(19) United States
(12) Patent Application Publication
(10) Pub. No.: US 2022/0087537 A1
(43) Pub. Date: Mar. 24, 2022

(54) WVSM With Continuous Monitoring and Automatic Acquisition of Selected Signals

(71) Applicant: West Affum Holdings Corp., Grand Cayman (KY)

(72) Inventors: Zoie R. Engman, Kirkland, WA (US); David P. Finch, Bothell, WA (US); Joseph L. Sullivan, Kirkland, WA (US)

(73) Assignee: West Affum Holdings Corp., Grand Cayman (KY)

(21) Appl. No.: 17/481,032

(22) Filed: Sep. 21, 2021

Related U.S. Application Data
(60) Provisional application No. 63/081,087, filed on Sep. 21, 2020.

(51) Int. Cl. A61B 5/024; A61B 5/00; A61B 5/11; A61B 5/28; A61N 1/04; A61N 1/39

(52) U.S. Cl. CPC A61B 5/024; A61B 5/746; A61B 5/1118; A61N 1/3904; A61B 5/28; A61B 5/6805; A61N 1/046; A61B 5/4848

(57) ABSTRACT

A wearable vital signs monitor (WVSM) comprises one or more sensors to obtain one or more patient signals measured from a patient, and a processor to receive the one or more measured patient signals. The processor is configured to analyze the one or more patient signals against one or more criteria sets, determine whether at least one of the criteria sets is met, and if at least criteria set is met, cause a requested action to be performed. The requested action can comprise a least one of obtaining one or more additional patient signal measurements as defined by a patient signal measurement set corresponding to the met criteria set or performing one or more alert actions according to the met criteria set.

### CROSS-REFERENCE TO RELATED APPLICATIONS

The present application claims the benefit of U.S. Provisional Application No. 63/081,087 (C00003641.USP1) filed Sep. 21, 2020. Said Application No. 63/081,087 is hereby incorporated herein by reference in its entirety.

### BACKGROUND

Wearable vital signs monitors (WVSMs) can provide benefits over non-wearable systems because such WVSMs can provide continuous 24/7 monitoring of key physiological signals. The accuracy of some physiologic signals, however, can be affected by the conditions under which they are sensed or collected. Furthermore, through study and analysis, it has been determined that acquisition of some physiological signals such as heart rate trend can be susceptible to noise or interference. Other signals such as resting heart rate can be difficult to measure unless the wearer has been sedentary for a certain period of time. Still other signals such as non-invasive blood pressure monitoring cannot be measured accurately unless the wearer is not moving to improve accuracy. Further still, some signals such as body weight cannot be measured with a WVSM and may involve using additional equipment such as a that is not a part of the core wearable system. Additionally, current wearable vital signs monitors are not capable of receiving inputs of variable types from multiple sources, processing the signals, and in response taking one or more actions based on the received inputs according to multiple criteria sets.

### DESCRIPTION OF THE DRAWING FIGURES

Claimed subject matter is particularly pointed out and distinctly claimed in the concluding portion of the specification. However, such subject matter may be understood by reference to the following detailed description when read with the accompanying drawings in which:

FIG. 1 is a diagram of an example of a wearable vital signs monitor (WVSM) to be worn by a patient in accordance with one or more embodiments.

FIG. 2 is a diagram of an example wearable patient monitoring system (WPMS) including an Auto Acquisition Engine (AAE) in accordance with one or more embodiments.

FIG. 3 is a diagram of an example wearable cardioverter defibrillator (WCD) including the Auto Acquisition Engine (AAE) of FIG. 2 in accordance with one or more embodiments.

FIG. 4 is a diagram of an example method executable by the Auto Acquisition Engine (AAE) of FIG. 2 in accordance with one or more embodiments.

FIG. 5 is a diagram of sample embodiments of components of a wearable vital signs monitor (WVSD) comprising a WCD system in accordance with one or more embodiments.

It will be appreciated that for simplicity and/or clarity of illustration, elements illustrated in the figures have not necessarily been drawn to scale. For example, the dimensions of some of the elements may be exaggerated relative to other elements for clarity. Further, if considered appropriate, reference numerals have been repeated among the figures to indicate corresponding and/or analogous elements.

### DETAILED DESCRIPTION

In the following detailed description, numerous specific details are set forth to provide a thorough understanding of claimed subject matter. It will, however, be understood by those skilled in the art that claimed subject matter may be practiced without these specific details. In other instances, well-known methods, procedures, components and/or circuits have not been described in detail.

In the following description and/or claims, the terms coupled and/or connected, along with their derivatives, may be used. In particular embodiments, connected may be used to indicate that two or more elements are in direct physical and/or electrical contact with each other. Coupled may mean that two or more elements are in direct physical and/or electrical contact. Coupled however, may also mean that two or more elements may not be in direct contact with each other, but yet may still cooperate and/or interact with each other. For example, “coupled” may mean that two or more elements do not contact each other but are indirectly joined together via another element or intermediate elements. Finally, the terms “on,” “overlying,” and “over” may be used in the following description and claims. “On,” “overlying,” and “over” may be used to indicate that two or more elements are in direct physical contact with each other. It should be noted, however, that “over” may also mean that two or more elements are not in direct contact with each other. For example, “over” may mean that one element is above another element but not contact each other and may have another element or elements in between the two elements. Furthermore, the term “and/or” may mean “and”, it may mean “or”, it may mean “exclusive-or”, it may mean “one”, it may mean “some, but not all”, it may mean “neither”, and/or it may mean “both”, although the scope of claimed subject matter is not limited in this respect. In the following description and/or claims, the terms “comprise” and “include,” along with their derivatives, may be used and are intended as synonyms for each other.

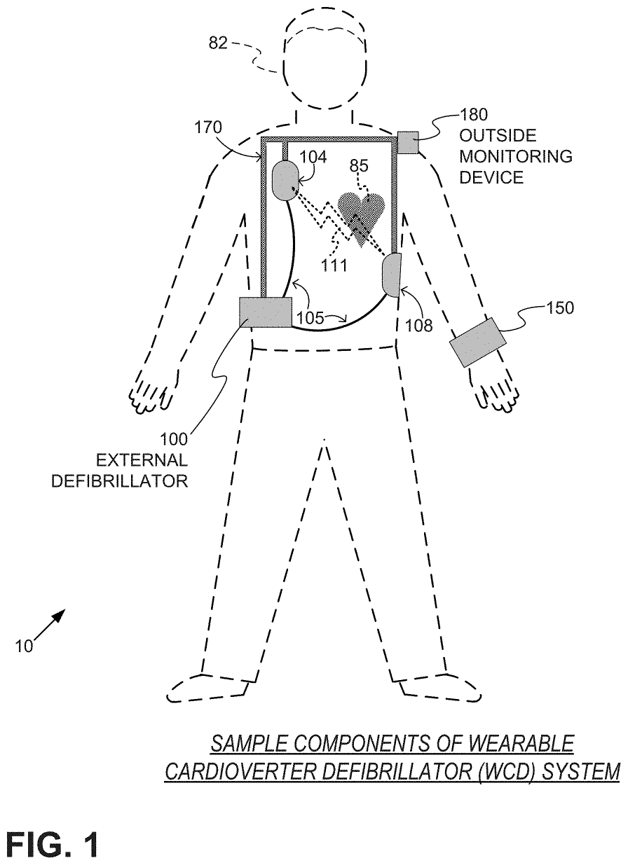

Referring now to FIG. 1, a diagram of an example of a wearable vital signs monitor (WVSM) to be worn by a patient in accordance with one or more embodiments will be discussed. FIG. 1 shows a wearable cardioverter defibrillator (WCD) system as one example embodiment of a WVSM 10 being worn by a patient 82. It should be noted that a WCD system is only one example of a WVSM 10, wherein the WVSM 10 can be embodied as other systems or devices, and the scope of the disclosed subject matter is not limited in this respect. In this example, the WVSM 10 includes an external defibrillator or monitor module 100 that is coupled to a support structure 170, which can be a garment, a harness, a belt, a shirt, a vest, etc. The support structure 170 may have one or more sensors (e.g., sensors 104 and 108) attached to or integrated into the support structure 170. These sensors are communicatively coupled to the external monitor 100 via leads or cables 105. Although only two sensors are shown, in other embodiments there may be more than two sensors. The sensors can be configured to monitor the vital signs of the patient 82. For example, some of the sensors may be electrocardiogram (ECG) sensors that can monitor the heart rate and optionally other physiological parameters of the patient's heart 85.

In some embodiments, one or more outside monitoring devices 180 may be attached to or communicatively coupled to the support structure. For example, outside monitoring device 180 may be any type of wearable device such as a pulse oximeter module, fitness tracker, blood pressure monitor, glucose monitor, etc. that communicates with but is not included in the external monitor 100. In some embodiments, outside monitoring device 180 can include, comprise, or communicate with a wearable monitor or sensor 150, for example a device that is worn on a limb of the patient 82, or on some other location of the patient's body. In some examples, a wearable vital signs monitor can be realized or implemented in whole or in part in the wearable monitor or sensor 150, in whole or in part in outside monitoring device 180, or in whole or in part in external defibrillator 100. Thus, in general a wearable vital signs monitor can refer to the overall WVSM 10 system, external defibrillator 100, outside monitoring device 180, or wearable monitor sensor 150, or any combination thereof, and the scope of the disclosed subject matter is not limited in this respect. For purposes of discussion, WVSM 10 can refer to the wearable vital signs monitor.

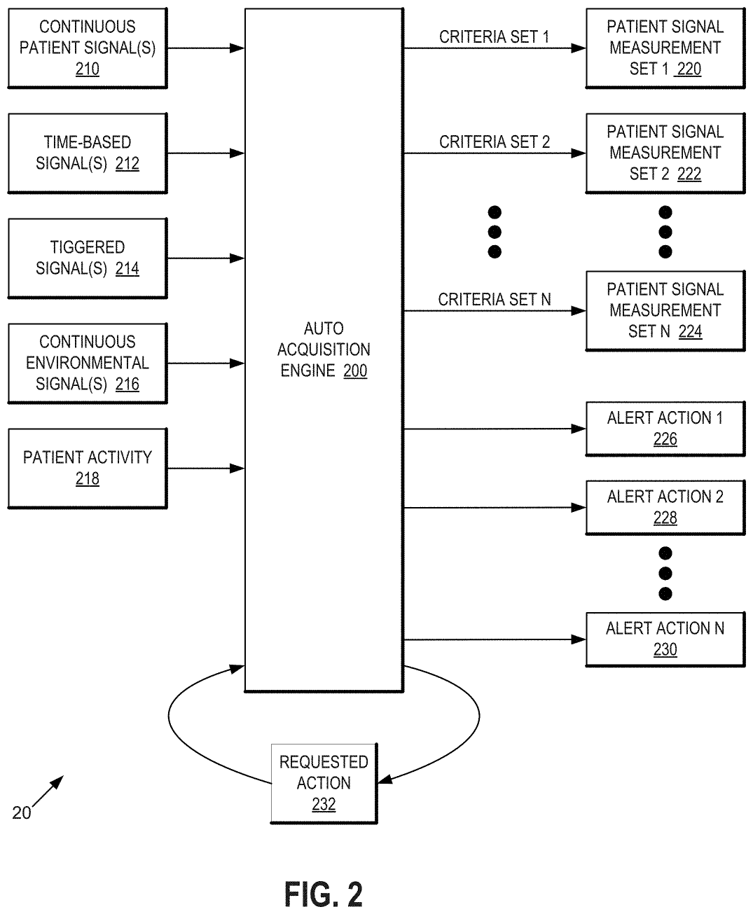

In one or more embodiments, WVSM 10 as shown in FIG. 1 can provide enhancements to the WVSM disclosed in US Patent Application publication US 2020/0281479 A1 which is hereby incorporated herein by reference, for example by implementing an Auto Acquisition Engine (AAE), for example as shown in further detail in FIG. 2 below, in the external monitor 100. The Auto Acquisition Engine can be configured to receive inputs from multiple sensors and of multiple types of signals including non-patient physiological signals such environmental signals, time signals, user interface signals, patient activity signals such as driving, playing video games, sleeping, and so on. Based on analysis of the inputs, the Auto Acquisition Engine can take different actions such as monitor different and/or additional patient parameters, issue prompts and/or alerts to the patient to take certain actions, for example “adjust” sensors, attach other sensors such as a blood pressure (BP) monitor, a weight scale, glucose monitor, pulse oximeter, etc., stop moving to reduce ECG noise, drink liquids, take medication, call for assistance, and so on. The analysis of the inputs to the Auto Acquisition Engine can be used for identifying different conditions such as heart failure, stroke, diabetic conditions, concussion, disorientation, syncope, and so on. Such an Auto Acquisition Engine is shown in and discussed with respect to FIG. 2, below.

Referring now FIG. 2, a diagram of an example wearable patient monitoring system (WPMS) including an Auto Acquisition Engine (AAE) in accordance with one or more embodiments will be discussed. In some embodiments, the wearable patient monitoring system (WPMS) 20 comprises a wearable vital signs monitor (WVSM) such as WVSM 10 of FIG. 1, although the scope of the disclosed subject matter is not limited in this respect. In accordance with embodiments of the present disclosure, WPMS 20 can include an auto acquisition engine (AAE) 200 that is configurable to process received input signals from one or more sensors and/or user interfaces, determine whether the input signals meet a criteria set, and cause the WPMS 20 to take one or more actions such as, for example, measure additional patient signals (e.g., vital signs), issue alerts or alarms, prompt the patient 82 to take an action, etc. In some cases, the requested action 232 can result in additional inputs being made to the AAE 200 as shown at the bottom of FIG. 2.

In the example of FIG. 2, WPMS 20 can include an auto acquisition engine (AAE) 200 that is configurable to receive one or more of: continuously monitored patient signals 210, time-based/periodically monitored patient signals 212, triggered signal(s) 214, continuously monitored environmental signals 216, and patient activity signals 218. Note, in some embodiments, the sensors detecting or receiving such signals can be from an implanted device, such as an implantable cardioverter defibrillator (ICD) or pacemaker.

Continuously monitored patient signals 210 can refer to patient signals that are monitored substantially continuously while the patient 82 is wearing the WPMS 20. In some embodiments, continuously monitored patient signals 210 can include one or more of: ECG, heart rate, step count (e.g., similar to or the same as the step counters in fitness trackers), patient temperature, respiration rate, and so on. In some embodiments, the WPMS 20 includes a wearable non-invasive blood pressure (NIBP) monitor such as wrist worn monitors proving measurements at a relatively fast rate, for example every five minutes, wearable blood pressure monitors based on pulse wave velocity sensing such as disclosed in US Patent Application publications US 2019/0125191 A1, US 2019/0053779 A1, or US 2019/0046152 A1 which are hereby incorporated herein by reference and which can be used to provide continuous blood pressure monitoring.

Time-based Patient signals 212 can refer to patient signals that are measured on a time-based or periodic manner. For example, the WPMS 20 can have inputs for a pulse oximeter that the patient wears while sleeping, or measures the patient's core temperature on a periodic basis, for example hourly.

Triggered signals 214 can refer to signals that the patient initiates by coupling a sensor device to the WPMS 20 or operating a user interface of the WPMS 20. For example, a diabetic patient may couple a glucose tester, wired or wirelessly, to the WPMS 20 to input blood glucose measurements, a weight scale to input the patient's body weight, a camera that monitors skin color to detect patient physiological parameters such as disclosed in US Patent Application publication US 2014/0078301 A1 which is hereby incorporated herein by reference, or a voice command using for example an intelligent voice responsive assistant or artificial intelligence (AI) agent to the WPMS 20 to initiate a patient signal measurement. In some embodiments, the WPMS 20 is configurable to enable a patient 82 to enter a self-assessment such as “I am feeling dizzy”, “I am experiencing chest pain”, and so on, via a user interface of the WPMS 20.

Continuously monitored environmental signals 216 can refer to signals that are monitored substantially continuously that are related to the environment in which the patient 82 is located. For example, the WPMS 20 can have temperature, altitude, Global Positioning System (GPS), and/or humidity sensors. In some embodiments in which the WPMS 20 has a wireless data connection, for example a wireless local area network (WLAN) or wireless wide area network (WWAN) connection, the WPMS 20 can retrieve weather, air quality, and/or pollen count information for the WPMS's location, for example determined by GPS or by a network identifier for a base station or cell to which the WPMS is connected, on a regular basis such as every five minutes to provide a relatively continuous monitoring of these environmental parameters.

Example input signals are summarized in Table 1 below according to some embodiments. In some other embodiments, one or more of the listed signals may be listed in other columns than as shown in Table 1 below. For example, Time-based Patient signals such as blood pressure and blood oxygen saturation (SpO2) may be listed in the Triggered Patient signals column in which the patient 92 would couple a blood pressure monitor or pulse oximeter to the WPMS 20, which would trigger the measurement.

TABLE 1

| | | | Continuous | |
|---|---|---|---|---|
| Continuous | Time-based | Triggered | Environ. | Patient |
| Patient Signals | Patient Signals | Signals | Signals | Activity |
| ECG | Body position | Body weight | Ambient temp. | Walking |
| | | | | detect. |
| Heart rate | Hemodynamic | Blood glucose | GPS | Sleeping |
| | | | | detect. |
| AF Burden | Blood pressure | Blood alcohol | Air Quality | Unsteady gait |
| Step count | Water weight % | Skin color | Altitude | Sitting detect. |
| Respiration rate | Heart sounds | ETCO2 | Humidity | Driving |
| | | | | vehicle |
| Core body temp. | SpO2 | Man. Edema test | Pollen count | Riding bicycle |
| Extremity temp. | | Self- | CO2 level | Watching TV |
| | | Assessment | | |
| | | | CO level | Removed |
| | | | | WMPS |

In some embodiments, the AAE 200 is configurable to analyze the received input signals and determine whether the signals meet any of one or more Criteria Sets wherein which each Criteria Set can have different criteria for subsets of the inputs to the AAE 200 as described above. For example, Criteria Set 1 can include ranges for a first subset of the input signals, Criteria Set 2 can include ranges for a second, different subset of the input signals, and so on up to Criteria Set N. In some embodiments, a first Patient Signal Measurement Set 1 220 can be used for Criteria Set 1, a second Patient Signal Measurement Set 222 can be used for Criteria Set 2 222, and so on, up to Criteria Set N being used for Patient Signal Measurement Set N 225. In some embodiments, when the AAE 200 determines that one of the Criteria sets has been satisfied, the AAE 200 is configurable to cause the WPMS 20 to measure one or more additional patient signals as defined in the Patient Signal Measurement Sets, or an Alert Action as described further below. For example, Criteria Set 1 can be set to monitor a patient 82 susceptible to bradycardia by setting a heart rate threshold below which the Criteria Set 1 is met. The AAE 200 then can cause the WPMS 20 to take one or more additional patient signal measurements as specified in Patient Signal Measurement Set 1 220. Criteria Sets may be defined for different conditions such as heart failure decompensation, myocardial infarction (MI), sudden cardiac arrest (SCA), atrial fibrillation (AF), high blood pressure, syncope, stroke, diabetes, post-traumatic stress disorder (PTSD), anxiety, depression, concussion, drug overdose, inebriation, medication compliance, drug interactions, and so on.

In some embodiments, the AAE 200 can be configurable to define one or more Patient Signal Measurement Sets, in which each set specifies different sets of additional patient signal measurements to be performed in response to a particular Criteria Set being met. For example, in the previously described bradycardia example, Patient Signal Measurement Set 1 may specify a self-assessment and blood pressure and SpO2 measurements when Criteria Set 1 is met. Patient Signal Measurement Sets may be defined for different conditions such as heart failure decompensation, MI, SCA, AF, high blood pressure, syncope, stroke, diabetes, PTSD, anxiety, depression, concussion, drug overdose, inebriation, medication compliance, drug interactions, and so on.

In some embodiments, the AAE 200 can be configurable to define one or more Alert Actions to be performed by the WPMS 20 in response to a particular Criteria Set being met. The one or more Alert Actions can comprise a first Alert Action 1 226, a second Alert Action 2 228, up to Alert Action N 230. In some examples, an Alert Action can be an alert or prompt for the patient to remain still so that a more accurate ECG can be taken in response to a Criteria Set defined for arrhythmia detection. In another example, an Alert Action can be associated with a Criteria Set defined for high heart rate and physical activity detection, prompting the patient 82 to reduce the activity level until the heart rate reaches a safer level. In yet another example, an Alert Action can be associated with a Criteria Set defined for disorientation, for example in response to medication, alerting the patient 82 to avoid driving or operating machinery. Other actions can include prompts to connect, reconnect, and/or adjust a sensor, consult with a nurse or other medical practitioner, call 911 or emergency services, take a medication, and so on. Alert Actions can be defined for different conditions such as heart failure decompensation, MI, SCA, AF, high blood pressure, syncope, stroke, diabetes, PTSD, anxiety, depression, concussion, drug overdose, inebriation, medication compliance, drug interactions, and so on.

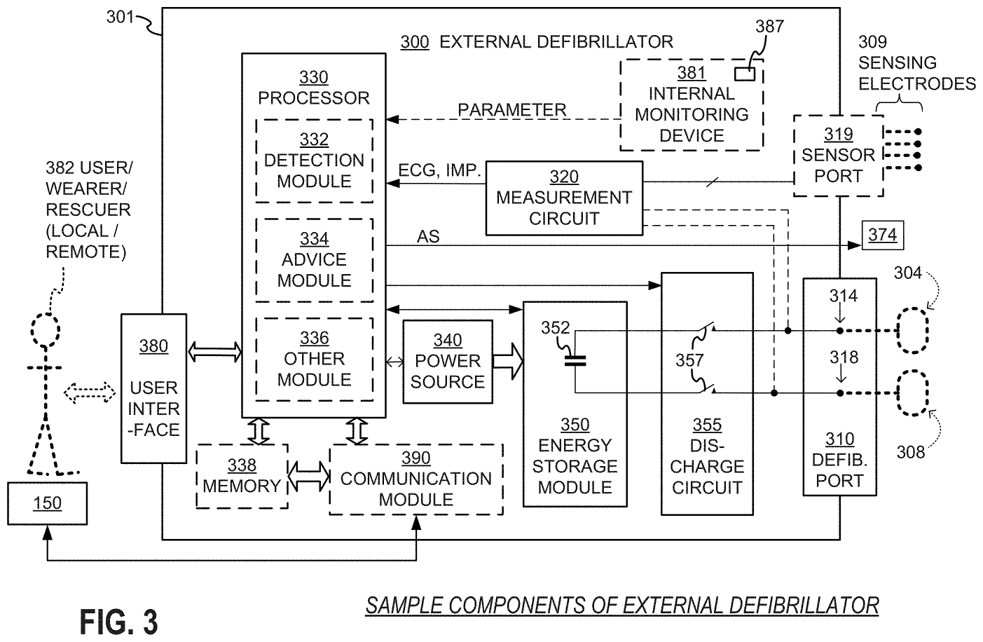

Referring now to FIG. 3, a diagram of an example wearable cardioverter defibrillator (WCD) including the Auto Acquisition Engine (AAE) of FIG. 2 in accordance with one or more embodiments will be discussed. In some embodiments, one or more therapy modules such as pacing or defibrillation can be added to the WVSM 10 of FIG. 1 to implement a type of wearable cardioverter defibrillator (WCD) or external pacer. FIG. 3 shows an example WCD with the AAE of FIG. 2 implemented in processor 330, for example as other module 336. It should be noted that some embodiments of a WPMS 20 or WVSM 10 can be similar to that shown in FIG. 3 with the components used only for delivering a shock, for example energy storage module 350, discharge circuit 355, defib. port 310, and so on, being omitted.

The components shown in FIG. 3 can be provided in a housing 301, which may also be referred to as casing 301. External defibrillator 300 is intended for a patient who would be wearing it, such as ambulatory patient 82 of FIG. 1. Defibrillator 300 further can include a user interface 380 for a user 382. User 382 can be patient 82, also known as wearer 82. Alternatively, user 382 can be a local rescuer at the scene, such as a bystander who can offer help or assistance, or a trained person. In some examples, user 382 can be a remotely located trained caregiver in communication with the WCD system.

User interface 380 can be provided in a number of ways. User interface 380 can include output devices, which can be visual, audible, or tactile, for communicating to a user by outputting images, sounds or vibrations. Images, sounds, vibrations, and anything that can be perceived by user 382 can also be called human-perceptible indications (HPIs). There are many examples of output devices. For example, an output device can be a light, or a screen to display what is sensed, detected and/or measured, and provide visual feedback to rescuer 382 for their resuscitation attempts, and so on. Another output device can be a speaker, which can be configured to issue voice prompts, beeps, loud alarm sounds and/or words to warn bystanders, and so on.

User interface 380 further can include input devices for receiving inputs from users. Such input devices may include various controls, such as pushbuttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock.

Defibrillator 300 can include an internal monitoring device 381. Monitoring device 381 is called an “internal” device because it is incorporated within housing 201. Monitoring device 381 can sense or monitor patient parameters such as patient physiological parameters, system parameters and/or environmental parameters, all of which can be called patient data. In other words, internal monitoring device 381 can be complementary or an alternative to outside monitoring device 180 of FIG. 1. Allocating which of the parameters are to be monitored by which of monitoring devices 180 or 381 can be done according to design considerations. Monitoring device 381 can include one or more sensors, as also described elsewhere herein.

Patient parameters can include patient physiological parameters. Patient physiological parameters can include, for example and without limitation, those physiological parameters that can be of any help in detecting by the WCD system whether or not the patient 82 needs a shock or other intervention or assistance. Patient physiological parameters also optionally can include the patient's medical history, event history and so on. Examples of such parameters include the patient's ECG, blood oxygen level, blood flow, blood pressure, blood perfusion, pulsatile change in light transmission or reflection properties of perfused tissue, heart sounds, heart wall motion, breathing sounds, and/or pulse. Accordingly, monitoring devices 180 or 381 can include one or more sensors configured to acquire patient physiological signals. Examples of such sensors or transducers include one or more electrodes to detect ECG data, a perfusion sensor, a pulse oximeter, a device for detecting blood flow such as a Doppler device, a sensor for detecting blood pressure such as a cuff, an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of the description herein, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, the local parameter can be a trend that can be detected in a monitored physiological parameter of patient 382. A trend can be detected by comparing values of parameters at different times over short and long terms. Parameters whose detected trends can particularly help a cardiac rehabilitation program include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, carbon dioxide (CO2), or other parameters such as those mentioned above, f) respiratory function, respiratory rate, etc.; g) motion, level of activity; and so on. Once a trend is detected, it can be stored and/or reported via a communication link, along perhaps with a warning if warranted. From the report, a physician monitoring the progress of patient 382 will know about a condition that is either not improving or deteriorating.

Patient state parameters include recorded aspects of patient 382, such as motion, posture, whether the patient has spoken recently plus maybe also what they said, and so on, plus optionally the history of these parameters. Alternatively, one of these monitoring devices can include a location sensor such as a Global Positioning System (GPS) location sensor. Such a sensor can detect the location, plus a speed can be detected as a rate of change of location over time. Many motion detectors output a motion signal that is indicative of the motion of the detector, and thus of the patient's body. Patient state parameters can be very helpful in narrowing down the determination of whether sudden cardiac arrest (SCA) is indeed taking place.

A WCD system made according to embodiments discussed herein can include a motion detector. In such embodiments, a motion detector can be implemented within monitoring device 180 or monitoring device 381. Such a motion detector can be made in many ways as is known in the art, for example by using an accelerometer. In this example, a motion detector 387 can be implemented within monitoring device 381. A motion detector of a WCD system according to embodiments can be configured to detect a motion event. A motion event can be defined as is convenient, for example a change in motion from a baseline motion or rest, etc. In such cases, a sensed patient parameter is motion. System parameters of a WCD system can include system identification, battery status, system date and time, reports of self-testing, records of data entered, records of episodes and intervention, and so on. In response to the detected motion event, the motion detector can render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if monitoring device 180 or 381 includes a GPS location sensor as per the above, and if it is presumed that the patient is wearing the WCD system.

Defibrillator 300 typically includes a defibrillation port 310, which can be a socket in housing 301. Defibrillation port 310 includes electrical nodes 314 and 318. Leads of defibrillation electrodes 304 and 308, such as leads 105 of FIG. 1, can be plugged into defibrillation port 310, to make electrical contact with nodes 314 and 318, respectively. It is also possible that defibrillation electrodes 304 and 308 are connected continuously to defibrillation port 310 instead. Either way, defibrillation port 310 can be used for guiding, via electrodes, to the wearer at least some of the electrical charge that has been stored in an energy storage module 350 that is described more fully later herein. The electric charge can be the shock for defibrillation, pacing, and so on.

Defibrillator 300 can optionally also have a sensor port 319 in housing 301, which is also sometimes known as an ECG port. Sensor port 319 can be adapted for plugging in sensing electrodes 309, which are also known as ECG electrodes and ECG leads. It is also possible that sensing electrodes 309 can be connected continuously to sensor port 319, instead. Sensing electrodes 309 are types of transducers that can help sense an ECG signal, for example a 12-lead signal, or a signal from a different number of leads, especially if the leads make good electrical contact with the body of the patient 382 and in particular with the skin of the patient 382. As with defibrillation electrodes 304 and 308, the support structure 170 can be configured to be worn by patient 382 to maintain sensing electrodes 309 on a body of patient 382. For example, sensing electrodes 309 can be attached to the inside of support structure 170 for making good electrical contact with the patient, similarly with defibrillation electrodes 304 and 308.

Optionally, a WCD system according to embodiments also includes a fluid that can be deployed automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid can be in the form of a low-viscosity gel so that the fluid does not flow away after being deployed from the location it is released near the electrode. The fluid can be used for both defibrillation electrodes 304 and 308, and for sensing electrodes 309.

The fluid may be initially stored in a fluid reservoir (not shown in FIG. 3). Such a fluid reservoir can be coupled to the support structure 170. In addition, a WCD system according to some embodiments further includes a fluid deploying mechanism 374. Fluid deploying mechanism 374 can be configured to cause at least some of the fluid to be released from the reservoir and be deployed near one or both of the patient locations to which electrodes 304 and 308 are configured to be attached to the patient 382. In some embodiments, fluid deploying mechanism 374 can be activated prior to the electrical discharge responsive to receiving activation signal (AS) from processor 330, which is described more fully later herein.

In some embodiments, defibrillator 300 can also include a measurement circuit 320, as one or more of its working together with its sensors or transducers. Measurement circuit 320 senses one or more electrical physiological signals of the patient 382 from sensor port 319, if provided. Even if defibrillator 300 lacks sensor port 319, measurement circuit 320 can optionally obtain physiological signals through nodes 314 and 318 instead, when defibrillation electrodes 304 and 308 are attached to the patient 382. In these cases, the input reflects an ECG measurement. The patient parameter can be an ECG, which can be sensed as a voltage difference between electrodes 304 and 308. In addition, the patient parameter can be an impedance, which can be sensed between electrodes 304 and 308 and/or between the connections of sensor port 219 considered pairwise. Sensing the impedance can be useful for detecting, among other things, whether these electrodes 304 and 308 and/or sensing electrodes 309 are not making good electrical contact with the patient's body. These patient physiological signals can be sensed when available. Measurement circuit 320 can then render or generate information about them as inputs, data, other signals, etc. As such, measurement circuit 320 can be configured to render a patient input responsive to a patient parameter sensed by a sensor. In some embodiments, measurement circuit 320 can be configured to render a patient input, such as values of an ECG signal, responsive to the ECG signal sensed by sensing electrodes 309. More strictly speaking, the information rendered by measurement circuit 320 is output from it, but this information can be called an input because it is received as an input by a subsequent device or functionality.

Defibrillator 300 also includes a processor 330. Processor 330 can be implemented in a number of ways in various embodiments. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can include, or have access to, a non-transitory storage medium, such as memory 338 that is described more fully later herein. Such a memory 338 can have a non-volatile component for storage of machine-readable and machine-executable instructions. A set of such instructions can also be called a program. The instructions, which can also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described herein.

Processor 330 can be considered to have a number of modules. One such module can be a detection module 332. Detection module 332 can include a Ventricular Fibrillation (VF) detector. The patient's sensed ECG from measurement circuit 320, which can be available as inputs, data that reflect values, or values of other signals, may be used by the VF detector to determine whether the patient is experiencing VF. Detecting VF can be useful because VF typically results in SCA. Detection module 332 can also include a Ventricular Tachycardia (VT) detector, and so on.

Another such module in processor 330 can be an advice module 334, which generates advice for what to do. The advice can be based on outputs of detection module 332. There can be many types of advice according to embodiments. In some embodiments, the advice is a shock/no shock determination that processor 330 can make, for example via advice module 334. The shock/no shock determination can be made by executing a stored Shock Advisory Algorithm. A Shock Advisory Algorithm can make a shock/no shock determination from one or more ECG signals that are captured according to embodiments and determine whether or not a shock criterion is met. The determination can be made from a rhythm analysis of the captured ECG signal or otherwise.

In some embodiments, when the determination is to shock, an electrical charge is delivered to the patient. Delivering the electrical charge is also known as discharging and shocking the patient. As mentioned above, such can be for defibrillation, pacing, and so on.

In ideal conditions, a very reliable shock/no shock determination can be made from a segment of the sensed ECG signal of the patient. In practice, however, the ECG signal often can be corrupted by electrical noise, which makes it difficult to analyze. Too much noise sometimes causes an incorrect detection of a heart arrhythmia, resulting in a false alarm to the patient. Noisy ECG signals may be handled as described in U.S. patent application Ser. No. 16/037,990 filed Jul. 17, 2018 and published as US 2019/0030351 A1, and also in U.S. patent application Ser. No. 16/038,007 filed on Jul. 17, 2018 and published as US 2019/0030352 A1, both by the same applicant and both hereby incorporated herein by reference.

Processor 330 can include additional modules, such as other module 336, for other functions. In addition, if internal monitoring device 381 is indeed provided, processor 330 can receive its inputs, and so on.

Defibrillator 300 optionally further includes a memory 338 which can work together with processor 330. Memory 338 can be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. Memory 338 therefore can comprise a non-transitory storage medium. Memory 238, if provided, can include programs for processor 330, which processor 330 can be able to read and execute. More particularly, the programs can include sets of instructions in the form of code, which processor 330 can be able to execute upon reading. The programs can also include other information such as configuration data, profiles, scheduling etc. that can be acted on by the instructions. Executing is performed by physical manipulations of physical quantities, and may result in functions, operations, processes, acts, actions and/or methods to be performed, and/or the processor 330 to cause other devices or components or blocks to perform such functions, operations, processes, acts, actions and/or methods. The programs can be operational for the inherent needs of processor 330, and can also include protocols and ways that decisions can be made by advice module 334. In addition, memory 338 can store prompts for user 382, for example when the user is a local rescuer. Moreover, memory 338 can store data. This data can include patient data, system data and environmental data, for example as learned by internal monitoring device 381 and outside monitoring device 180. The data can be stored in memory 338 before it is transmitted out of defibrillator 300, or be stored there after it is received by defibrillator 300.

Defibrillator 300 can optionally include a communication module 390, for establishing one or more wired or wireless communication links with other devices of other entities, such as a remote assistance center, Emergency Medical Services (EMS), and so on. The communication links can be used to transfer data and commands. The data may be patient data, event information, therapy attempted, cardiopulmonary resuscitation (CPR) performance, system data, environmental data, and so on. For example, communication module 390 may transmit wirelessly, for example on a daily basis, heart rate, respiratory rate, and other vital signs data to a server accessible over the internet, for instance as described in U.S. Patent Application publication US 2014/0043149 A1, entitled “MOBILE COMMUNICATION DEVICE & APP FOR WEARABLE DEFIBRILLATOR SYSTEM”, which is hereby incorporated herein by reference. This data can be analyzed directly by the patient's physician and can also be analyzed automatically by algorithms designed to detect a developing illness and then notify medical personnel via text, email, phone, and so on. Module 390 can also include such interconnected sub-components as may be deemed necessary by a person skilled in the art, for example an antenna, portions of a processor, supporting electronics, outlet for a telephone or a network cable, and so on. In some examples, wearable sensor 150 as shown in FIG. 1 can communicate with communication module 390 of defibrillator 300, for example via a wired or a wireless connection.

Defibrillator 300 can also include a power source 340. To enable portability of defibrillator 300, power source 340 typically includes a battery. Such a battery is typically implemented as a battery pack, which can be rechargeable or not. Sometimes a combination is used of rechargeable and non-rechargeable battery packs. Other embodiments of power source 340 can include an alternating-current (AC) power override, for where AC power will be available, an energy-storing capacitor, and so on. Appropriate components may be included to provide for charging or replacing power source 340. In some embodiments, power source 340 can be controlled and/or monitored by processor 330.

Defibrillator 300 may additionally include an energy storage module 350. Energy storage module 350 can be coupled to the support structure of the WCD system, for example either directly or via the electrodes and their leads. Module 230 is where some electrical energy can be stored temporarily in the form of an electrical charge, when preparing it for discharge to administer a shock. In embodiments, module 350 can be charged from power source 340 to the desired amount of energy, as controlled by processor 330. In typical implementations, module 350 includes a capacitor 352, which can be a single capacitor or a system of capacitors, and so on. In some embodiments, energy storage module 350 includes a device that exhibits high power density, such as an ultracapacitor. As described above, capacitor 352 can store the energy in the form of an electrical charge, for delivering to the patient.

A decision to shock can be made responsive to the shock criterion being met, as per the above-mentioned determination. When the decision is to shock, processor 330 can be configured to cause at least some or all of the electrical charge stored in module 350 to be discharged through patient 82 while the support structure 170 is worn by patient 82, so as to deliver a shock 111 to patient 82.

For causing the discharge, defibrillator 300 moreover includes a discharge circuit 355. When the decision is to shock, processor 330 can be configured to control discharge circuit 2355 to discharge through the patient at least some of all of the electrical charge stored in energy storage module 350. Discharging can be to nodes 314 and 318, and from there to defibrillation electrodes 304 and 308, so as to cause a shock to be delivered to the patient 82. Circuit 355 can include one or more switches 357. Switches 357 can be realized in a number of ways, such as an H-bridge, and so on. Circuit 355 can also be controlled via processor 330, and/or user interface 380.

A time waveform of the discharge may be controlled by thus controlling discharge circuit 355. The amount of energy of the discharge can be controlled by how much energy storage module has been charged, and also by how long discharge circuit 355 is controlled to remain open. Defibrillator 300 can optionally include other components.

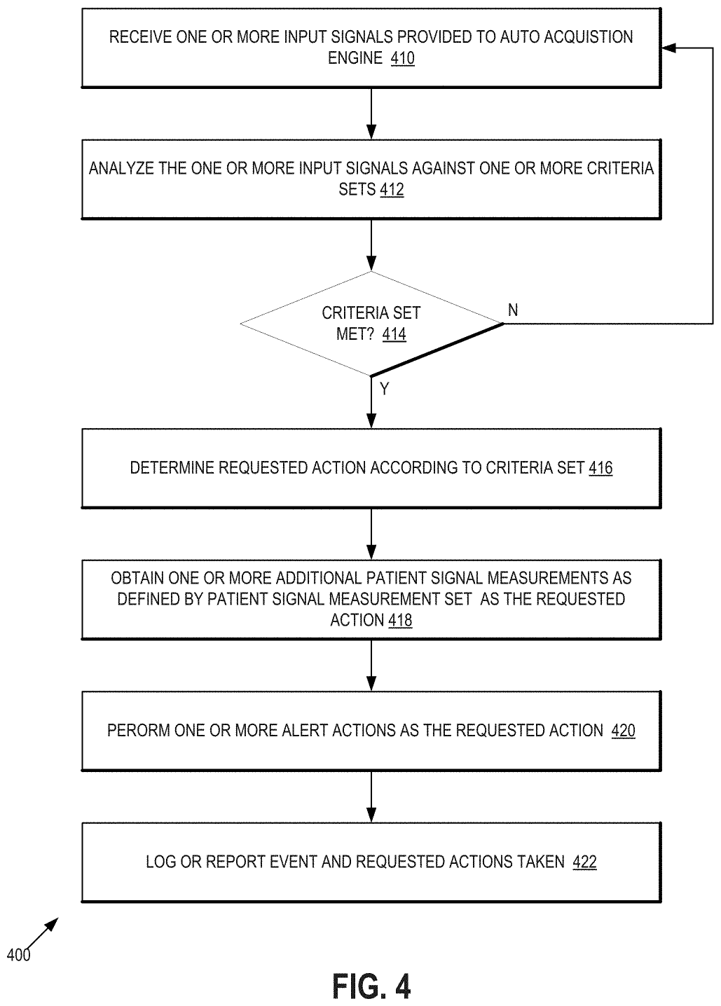

Referring now to FIG. 4, a diagram of an example method executable by the Auto Acquisition Engine (AAE) of FIG. 2 in accordance with one or more embodiments will be discussed. In some embodiments, method 400 of FIG. 4 can be executed by Auto Acquisition Engine 200 as part of a standalone patient monitoring system or wearable vital signs monitor (WVSM). In other embodiments, method 400 of FIG. 4 can be executed by a patient therapy module or device such as a pacer, defibrillator, automated external defibrillator (AED), or wearable cardioverter defibrillator (WCD) such as external defibrillator 300 of FIG. 3. It should be noted, however, that these are merely example embodiments of devices that are capable of executing method 400 of FIG. 4, and the scope of the disclosed subject matter is not limited in these respects. Furthermore, although FIG. 4 shows one particular number of operations and order of operations for method 400, method 400 can include more or fewer operations in various other orders, and the scope of the disclosed subject matter is not limited in these respects.

At operation 410 of method 400, auto acquisition engine 200 can receive one or more input signals provided to the auto acquisition engine 200. The one or more input signals can include, for example continuous patient signals 210, time-based signals 212, triggered signals 214, continuous environmental signals 216, or patient activity 218 such as shown in and described with respect to FIG. 2 above or as outlined in Table 1 above. Auto acquisition engine 200 can then analyze at operation 412 the one or more received input signals against one or more Criteria Sets as described with respect to FIG. 2 above. A determination can be made at decision operation 414 whether one or more Criteria Sets have been met as a result of the analysis performed at operation 412. If no Criteria Sets have been met, then method 400 can continue at operation 401. If a Criteria Set is met, the requested action for the given Criteria Set can be determined at operation 416. For example, the requested action can include causing Wearable Patient Monitoring System (WPMS) 20 of FIG. 2 to measure one or more additional patient signals, or can include causing Wearable Patient Monitoring System (WPMS) 20 of FIG. 2 to perform one or more Alert Actions.

In one or more embodiments, the requested action can include obtaining one or more additional patient signal measurements at operation 418 as defined by a particular Patient Measurement Set corresponding to the Criteria Set being met at decision operation 414. For example, the AAE 200 can be configurable to define one or more Patient Signal Measurement Sets in which each set specifies different sets of additional patient signal measurements to be performed in response to a particular Criteria Set being met at operation 414. For example, Criteria Set 1 can be set to monitor a patient susceptible to bradycardia by setting a heart rate threshold below which the Criteria Set 1 is met as a determining factor for decision operation 414. Patient Signal Measurement Set 1 220 can then specify a self-assessment and blood pressure and SpO2 measurements when Criteria Set 1 is met at decision operation 414. Patient Signal Measurement Sets can be defined for different conditions such as heart failure decompensation, myocardial infarction (MI), sudden cardiac arrest (SCA), atrial fibrillation (AF), high blood pressure, syncope, stroke, diabetes, post-traumatic stress disorder (PTSD), anxiety, depression, concussion, drug overdose, inebriation, medication compliance, drug interactions, and so on.

In one more embodiments, the AAE 200 can be configurable to define one or more Alert Actions to be performed by the WPMS 20 at operation 420 in response to a particular Criteria Set being met at decision operation 414. For example, an Alert Action may be an alert or prompt for the patient to remain still so that a more accurate ECG may be taken in response to a Criteria Set defined for arrhythmia detection. Another example Alert Action can be associated with a Criteria Set defined for high heart rate and physical activity detection, prompting the patient to reduce the activity level until the heart rate reaches a safer level. Another example Alert Action may be associated with a Criteria Set defined for disorientation, for example in response to medication, alerting the patient to avoid driving or operating machinery. Other actions can include prompts to connect, reconnect, or adjust a sensor, consult with a nurse or other medical practitioner, call 911 or emergency services, take a medication, and so on. Alert Actions can be defined for different conditions such as heart failure decompensation, myocardial infarction (MI), sudden cardiac arrest (SCA), atrial fibrillation (AF), high blood pressure, syncope, stroke, diabetes, post-traumatic stress disorder (PTSD), anxiety, depression, concussion, drug overdose, inebriation, medication compliance, drug interactions, and so on.

Once the one or more additional patient signal measurements are obtained at operation 410, or the one or more alert actions are performed at operation 420, with either operation being performed or both operation being performed in response to a Criteria Set being met at decision operation 414, Wearable Patient Monitoring System (WPMS) 20 can log or report the event at operation 422 corresponding to the Criteria Set being met and the requested actions taken. Optionally, method 400 can continue to at operation 410 to monitor for the same Criteria Set being subsequent met, or for one or more other Criteria Sets being met, and the scope of the disclosed subject matter is not limited in these respects.

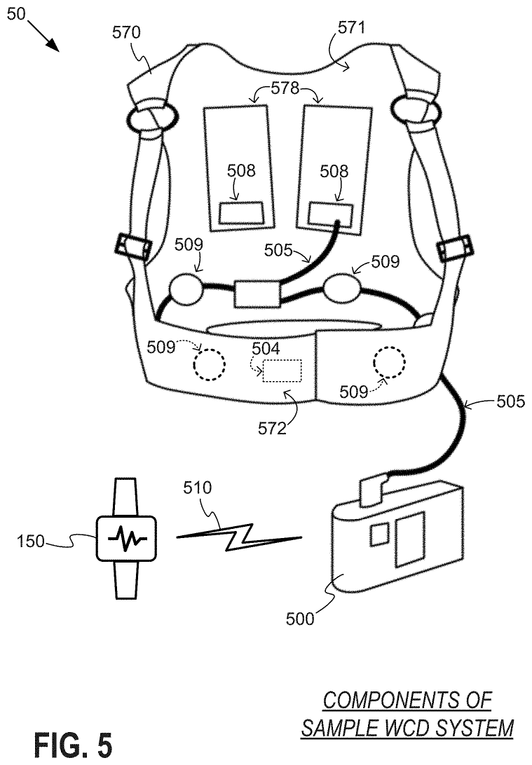

Referring now to FIG. 5, a diagram of sample embodiments of components of a wearable vital signs monitor (WVSD) comprising a WCD system in accordance with one or more embodiments will be discussed. WCD system 50 can comprise a support structure 570 that includes a vest-like wearable garment. Support structure 570 has a back side 571, and a front side 572 that closes in front of the chest of the patient 82. In some examples, wearable sensor 150 can operate as part of WCD system 50 as discussed in further detail below.

The WCD system 50 of FIG. 5 can include an external defibrillator 500. FIG. 5 does not show any support for external defibrillator 500, which can be carried in a purse, on a belt, by a strap over the shoulder, and so on. Wires 505 connect external defibrillator 500 to electrodes 504, 508, and/or 509. Of those, electrodes 504 and 508 are defibrillation electrodes, and electrodes 509 are ECG sensing electrodes.

Support structure 570 is configured to be worn by the ambulatory patient 82 to maintain electrodes 504, 508, and/or 509 on the body of the patient 82. Back defibrillation electrodes 508 can be maintained in pockets 578. The inside of pockets 578 can be made with loose netting, so that electrodes 508 can contact the back of the patient 82, especially with the help of the conductive fluid that has been deployed in such embodiments. In addition, sensing electrodes 509 can be maintained in positions that surround the patient's torso for sensing ECG signals and/or the impedance of the patient 82.

ECG signals in a WCD system 50 may include too much electrical noise to be useful. To ameliorate the problem, multiple ECG sensing electrodes 509 can be provided for presenting many options to processor 330 as shown in FIG. 3. These options comprise different vectors for sensing the ECG signal.

In accordance with one or more embodiments, wearable sensor 150 can communicate with external defibrillator 500, for example via a wireless communication link 510 in some embodiments. In other embodiments, wearable sensor 150 also can communicate with external defibrillator 500 via a wired communication link, and the scope of the disclosed subject matter is not limited in this respect. To realize a wearable vital signs monitor (WVSM) 10, the wearable patient monitoring system (WPMS) 20 of FIG. 2 including auto acquisition engine 200 can be implemented in whole or in part in defibrillator 500, for example as other module 336 of processor 330 as shown in FIG. 3, or in whole or in part in wearable sensor 150, and the scope of the disclosed subject matter is not limited in these respects.

Other embodiments can include combinations and sub-combinations of features described or shown in the drawings herein, including for example, embodiments that are equivalent to: providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

Although the claimed subject matter has been described with a certain degree of particularity, it should be recognized that elements thereof may be altered by persons skilled in the art without departing from the spirit and/or scope of claimed subject matter. It is believed that the subject matter pertaining to a wearable vital signs monitor with continuous monitoring and automatic acquisition of selected signals and many of its attendant utilities will be understood by the forgoing description, and it will be apparent that various changes may be made in the form, construction and/or arrangement of the components thereof without departing from the scope and/or spirit of the claimed subject matter or without sacrificing all of its material advantages, the form herein before described being merely an explanatory embodiment thereof, and/or further without providing substantial change thereto. It is the intention of the claims to encompass and/or include such changes.

What is claimed is:

1. A wearable vital signs monitor (WVSM), comprising:
one or more sensors to obtain one or more patient signals measured from a patient;
a processor to receive the one or more measured patient signals, wherein the processor is configured to:
analyze the one or more patient signals against one or more criteria sets;
determine whether at least one of the criteria sets is met; and
if at least one criteria set is met, cause a requested action to be performed, wherein the requested action comprises a least one of:
obtaining one or more additional patient signal measurements as defined by a patient signal measurement set corresponding to the met criteria set; or
performing one or more alert actions according to the met criteria set.

2. The WVSM of claim 1, wherein one or more of the patient signals comprises a continuously monitored patient signal that is monitored substantially continuously while the patient is using the WVSM.

3. The WVSM of claim 1, wherein one or more of the patient signals comprises a time-based patient signal that is measured on a time-based or periodic manner.

4. The WVSM of claim 1, wherein one or more of the patient signals comprises patient triggered signal initiated by the patient.

5. The WVSM of claim 1, wherein one or more of the patient signals comprises a continuously monitored environmental signal that is substantially continuously and related to an environment of the patient.

6. The WVSM of claim 1, wherein one or more of the patient signals comprises a patient activity signal that detects an activity engaged in by the patient.

7. The WVSM of claim 1, wherein a first criteria set comprises a first criterion for a first subset of the one or more patient signals, and a second criteria set comprises a second criterion for a second subset of the one or more patient signals.

8. The WVSM of claim 1, wherein a patient signal measurement set is defined for a first patient condition, and a patient signal measurement set is defined for a second patient condition.

9. The WVSM of claim 8, wherein the first patient condition or the second patient condition comprises at least one of heart failure decompensation, myocardial infarction (MI), sudden cardiac arrest (SCA), atrial fibrillation (AF), high blood pressure, syncope, stroke, diabetes, post-traumatic stress disorder (PTSD), anxiety, depression, concussion, drug overdose, inebriation, medication compliance, or drug interactions.

10. The WVSM of claim 1, wherein one or more of the alert actions comprises prompting the patient to perform a predetermined action in response to a corresponding one of the one or more criteria sets being met.

11. The WVSM of claim 10, wherein the patient is prompted to remain still, to reduce physical activity until a heart rate of the patient reaches a predetermined rate, to avoid driving or operating machinery, to connect, reconnect, or adjust a sensor, to consult with a medical practitioner, to call emergency services, or to take a medication.

12. The WVSM of claim 10, wherein one or more of the alert actions are defined for at least one of heart failure decompensation, myocardial infarction (MI), sudden cardiac arrest (SCA), atrial fibrillation (AF), high blood pressure, syncope, stroke, diabetes, post-traumatic stress disorder (PTSD), anxiety, depression, concussion, drug overdose, inebriation, medication compliance, or drug interactions.

13. The WVSM of claim 1, further comprising:
a support structure to be worn by the patient;
a plurality of electrocardiography (ECG) electrodes to provide an ECG signal to the processor as one or more of the patient signals, and a plurality of defibrillator electrodes, coupled with the support structure, to contact a skin of the patient when therapy is delivered to the patient; and
a defibrillator comprising a high voltage subsystem to provide a defibrillation voltage to the patient through the plurality of defibrillator electrodes in response to a shock signal received from the processor;
wherein the processor is configured to provide the shock signal to the high voltage subsystem when the criteria set is met.

14. A method executable by a wearable vital signs monitor, the method comprising:
obtaining one or more patient signals measured from a patient;
analyzing the one or more patient signals against one or more criteria sets;
determining whether at least one of the criteria sets is met; and
if at least criteria set is met, causing a requested action to be performed, wherein the requested action comprises a least one of:
obtaining one or more additional patient signal measurements as defined by a patient signal measurement set corresponding to the met criteria set; or
performing one or more alert actions according to the met criteria set.

15. The method of claim 14, wherein one or more of the patient signals comprises a continuously monitored patient signal that is monitored substantially continuously while the patient is using the WVSM.

16. The method of claim 14, wherein one or more of the patient signals comprises a time-based patient signal that is measured on a time-based or periodic manner.

17. The method of claim 14, wherein one or more of the patient signals comprises patient triggered signal initiated by the patient.

18. The method of claim 14, wherein one or more of the patient signals comprises a continuously monitored environmental signal that is substantially continuously and related to an environment of the patient.

19. The method of claim 14, wherein one or more of the patient signals comprises a patient activity signal that detects an activity engaged in by the patient.

20. The method of claim 14, wherein a first criteria set comprises a first criterion for a first subset of the one or more patient signals, and a second criteria set comprises a second criterion for a second subset of the one or more patient signals.
